(19) United States
(12) Patent Application Publication
(10) Pub. No.: US 2003/0039989 A1
(43) Pub. Date: Feb. 27, 2003

(54) Method for constructing an expression library and uses thereof

(76) Inventors: Steven Arthur Haney, Concord, MA (US); Piet A. J. de Boer, University Heights, OH (US)

Correspondence Address: Craig J. Arnold, Esq., Amster, Rothstein & Ebenstein, 90 Park Avenue, New York, NY, 10016, US

(21) Appl. No.: 10/024,659

(22) Filed: Dec. 17, 2001

Related U.S. Application Data
(60) Provisional application No. 60/281,076, filed on Apr. 3, 2001.

(51) Int. Cl. C12Q 1/68; C12N 1/18; C12N 15/74

(52) U.S. Cl. 435/6; 435/483; 435/254.2

(57) ABSTRACT

The present invention provides a method for constructing an expression library containing a gene that is toxic to bacteria, comprising the steps of: (a) isolating genomic DNA from a culture of cells of a first bacterium, wherein said genomic DNA contains a gene toxic to a second bacterium; (b) partially digesting said genomic DNA to produce genetic inserts, wherein at least one of the genetic inserts contains a gene to said second bacterium; (c) cloning said genetic inserts into a cloning vector; (d) ligating together, under suitable ligation conditions, said genetic inserts and said cloning vector, to produce ligation products; (e) transforming said ligation products directly into yeast; and (f) amplifying said ligation products in said yeast. Also provided are an expression library constructed in accordance with this method and a method for screening for a gene that is toxic to bacteria using said expression library.

### RELATED APPLICATIONS

This application claims the benefit of U.S. Provisional Application No. 60/281,076, filed Apr. 3, 2001.

### STATEMENT OF GOVERNMENT INTEREST

This invention was made with government support under NSF Grant No. MCB94-58197. As such, the United States government has certain rights in this invention.

### FIELD OF THE INVENTION

The present invention relates generally to the field of molecular biology, and, more particularly, to the construction of a new expression library containing cloned fragments of chromosomal DNA that code for genes that are toxic to bacteria. This library can be used to screen for, or to identify, antimicrobial agents. Accordingly, the present invention also provides methods for using the expression library, as disclosed herein, to screen for agents having antimicrobial activity.

### BACKGROUND OF THE INVENTION

Pathogens are microorganisms that have the capacity to cause disease. Bacteria and fungi account for most of the pathogens which produce infectious diseases in host organisms (20). In humans, bacterial infections manifest in many different disease states, and may be chronic and even life-threatening. Bacterial infections embrace such serious conditions as abnormalities of mental status (e.g., coma, convulsions); cardiac manifestations (e.g., failing myocardial function, tachycardia); endocrinologic dysfunctions (e.g., increased production of hormones, profound catabolism of muscle proteins); fever; hematologic manifestations (e.g., anemia, leukocytosis, thrombocytopenia); hepatic dysfunction; renal manifestations (e.g., acute renal failure, septic shock); respiratory manifestations (e.g., hyperventilation, respiratory alkalosis); and upper GI bleeding (20).

Antimicrobial agents act on bacterial pathogens and other microorganisms by interfering with important cellular processes, such as cell division, transcription, and translation. For example, antimicrobial agents may inhibit cell wall synthesis, activate enzymes that destroy the cell wall, increase cell membrane permeability, interfere with protein synthesis, or interfere with nucleic acid metabolism. Some antimicrobial agents, such as bacteriostatic drugs (e.g., azithromycin, chloramphenicol, erythromycin, and tetracyclines), slow microbial growth. Other antimicrobial agents, such as bactericidal drugs (e.g., aminoglycosides, cephalosporins, penicillins, polymyxins, and vancomycin), kill the targeted microorganism. To treat a bacterial infection, an antimicrobial agent must be toxic to the infectious bacterium, but not toxic to the patient. This selective toxicity generally is attained by ensuring that the antimicrobial agent is administered to the patient in an amount which may be tolerated by the patient (i.e., it does not deleteriously interfere with the host's biochemical system), but which is harmful or lethal to the bacterium (20).

Unfortunately, the development of resistance to antimicrobial agents is a problem that grows ever more widespread as strains of bacteria evolve in response to inappropriate or excessive use of such drugs. If a bacterium becomes resistant to a particular antimicrobial agent, it then becomes essential for a new antimicrobial agent to be identified. The problem of resistance to antimicrobial agents emphasizes the constant need to find replacement drugs. One means of identifying new antimicrobial agents consists of screening microbial genomes for toxic genes.

The yeast two-hybrid (Y2H) system is a versatile and powerful system for the identification of proteins that interact directly with a protein target (5, 10). It provides a useful method for screening gene libraries that contain cloned fragments of genetic material derived from a particular cell, tissue, or organism. The Y2H system has had a dramatic effect on studies of eukaryotic processes (e.g., signal transduction) and the cytoskeleton. However, due to the complexity of Y2H libraries, their handling and amplification is often made difficult. For example, representation of clones that express functional fusions can be lost if the fusion is expressed during amplification, or if it is somewhat toxic or growth inhibitory. Indeed, despite the success of expression libraries, several difficulties limit their broader application, and decrease the ease with which complete gene sequences may be obtained.

DNA libraries, other than yeast artificial chromosomes (YACs), are preserved in an E. coli host because of its high transformation efficiency. Most libraries have a eukaryotic origin, and toxicity is generally not considered a problem. Many of the protein families that are studied in the Y2H system are not found in E. coli. This suggests that fusions expressed within the E. coli host do not have targets with which they can interact. In addition, any toxicity that a clone may exhibit is essentially difficult to control. Finally, toxicity of some fusions is a small consequence of the power of the Y2H system. In contrast, toxicity is a greater problem for a two-hybrid system (or other expression library) of prokaryotic origin, since inappropriate expression of E. coli genes frequently occurs, particularly where the genes code for multisubunit complexes and transmembrane proteins.

### SUMMARY OF THE INVENTION

The present invention provides a method for constructing an expression library of bacterial genomic DNA, having increased complexity, that is stored in a yeast host strain, and that contains a gene that is toxic to bacteria. The method comprises the steps of: (a) isolating genomic DNA from a culture of cells of a first bacterium, wherein said genomic DNA contains a gene that is toxic to a second bacterium; (b) partially digesting said genomic DNA to produce genetic inserts, wherein at least one of said genetic inserts contains a gene that is toxic to said second bacterium; (c) cloning said genetic inserts into a cloning vector; (d) ligating together, under suitable ligation conditions, said genetic inserts and said cloning vector, to produce ligation products; (e) transforming said ligation products directly into yeast; and (f) amplifying said ligation products in said yeast. Also provided are an expression library constructed in accordance with this method and a method for screening for a gene that is toxic to bacteria using the expression library made in accordance with this method.

Additional objects of the present invention will be apparent in view of the description which follows.

### BRIEF DESCRIPTION OF THE FIGURES

FIG. 1 illustrates the sequence of the polylinker for plasmid pB42-C1. Restriction sites are underlined and labeled. The sequences of plasmids pB42-C2 and pB42-C3 are identical, except for the addition of one and two additional G residues, respectively, immediately prior to the Eco RI site, as indicated in the figure.

FIG. 2 depicts the characterization of clones from the yeast two-hybrid library. Plasmids recovered from yeast strain YM4271 were transformed into E. coli strain KC8 by electroporation. Transformants were selected by spotting the transformation mixture onto M9 plates that were supplemented (A) or not supplemented with tryptophan (B).

### DETAILED DESCRIPTION OF THE INVENTION

The present invention sets forth a novel method for constructing an expression library containing a gene that is toxic to bacteria. The inventors examined the steps involved in making a two-hybrid library, attempting to identify those steps that are particularly important for maintaining the complexity of a prokaryotic genomic library. They discovered that the most important factor is the avoidance of passaging the library through E. coli at any stage, such that compounds intrinsically toxic to E. coli are not selectively lost or reduced to such low levels that they can not be detected (e.g., by PCR).

The procedures used by the inventors in the construction of the expression library described herein were chosen to improve the likelihood that complexity of the library would be retained. Specifically, ligation products were transformed directly in yeast. This step was taken because it was clear to the inventors that many genes and gene fragments are toxic to E. coli when cloned on multicopy plasmids, even without strong prokaryotic promoters upstream of the open reading frames or gene fragments.

As a result of its novel construction, which avoids loss of functional clones that may be toxic to bacteria, the expression library of the present invention is more complex than other expression libraries. Thus, this library can facilitate identification and characterization of genes that are toxic to bacteria. In particular, it is expected that the expression library of the present invention will contain genes that are toxic to E. coli, B. subtilis, or another bacterium when inappropriately expressed (e.g., from a gene fusion). Such genes may play a role in controlling important cellular processes in bacteria. Examples of such important cellular processes in bacteria include, without limitation, cell division, transcription, and translation. Genes toxic to bacteria represent potential new targets for developing antimicrobial compounds.

The present invention provides a method for constructing an expression library containing a gene that is toxic to bacteria. The method comprises the following steps: (a) isolating genomic DNA from a culture of cells of a first bacterium, wherein said genomic DNA contains a gene that is toxic to a second bacterium; (b) partially digesting said genomic DNA to produce genetic inserts, wherein at least one of said genetic inserts contains a gene that is toxic to said second bacterium; (c) cloning said genetic inserts into a cloning vector; (d) ligating together, under suitable ligation conditions, said genetic inserts and said cloning vector, to produce ligation products; (e) transforming said ligation products directly into yeast; and (f) amplifying said ligation products in said yeast. Also provided is an expression library constructed in accordance with the method of the present invention.

Genomic DNA may be obtained from any bacterium, and may contain genes toxic to any bacterium. In a particular embodiment of the present invention, the first bacterium is Bacillus subtilis, and the second bacterium is Escherichia coli. In another embodiment of the present invention, the first bacterium is E. coli and the second bacterium is B. subtilis. It is also possible for the first bacterium and the second bacterium to be obtained from the same species or same strain. Any strains of E. coli (e.g., MG1655, DM1, KC8), B. subtilis (e.g., strain 168), and yeast (e.g., laboratory strains of Saccharomyces cerevisiae, such as EGY48 and YM4271) are appropriate for use in the present invention. Yeast strains EGY48 and YM4271, and E. coli strain KC8, may be obtained from Clontech. E. coli strain DM1 may be obtained from GibCo. E. coli strain MG1655 and B. subtilis strain 168 are general laboratory strains that may be obtained from ATCC.

In the method of the present invention, genomic DNA (e.g., chromosomal DNA) may be isolated from a cell culture using known methods. For example, it may be suitable to pellet and wash a cell culture with TES buffer (Tris-HCl, EDTA, and NaCl), pellet the cells again, resuspend the cells in sucrose and Tris, incubate the cells at an appropriate temperature and for an appropriate time (e.g., on ice, for 20 min), add a solution of lysozyme and EDTA, add a lysis solution (e.g., sarkosyl, Tris, and EDTA), add a solution of proteinase K, and then incubate at an appropriate temperature and for an appropriate time (e.g., at 55° C., for 60 min). The sample then may be extracted (e.g., with TE-saturated phenol, followed by PCI (TE-saturated phenol, chloroform, and isoamyl alcohol; 25:24:1), and the isolated DNA recovered.

Alternatively, as described herein, it may be suitable to wash a cell culture in EET (EDTA, EGTA, and Tris), resuspend the cells in EET, add a solution of lysozyme and RNase A, incubate at an appropriate temperature and for an appropriate time (e.g., at 37° C., for 30 min), add EET along with SDS and proteinase K, further incubate at an appropriate temperature and for an appropriate time (e.g., at 65° C., for 10 min), and then add NaCl, followed by a CTAB/NaCl solution. The solution then may be extracted (e.g., with PCI), and the aqueous phase transferred to a new tube. It then may be appropriate to add isopropanol, incubate the mixture at an appropriate temperature and for an appropriate time (e.g., at room temperature, for 15 min), then centrifuge at an appropriate speed and for an appropriate duration (e.g., at 15K rpm, for 20 min). The pellet then may be washed (e.g., with ethanol), recentrifuged, and resuspended (e.g., in TE) to recover isolated DNA.

For preparing genomic inserts for use in the present invention, isolated genomic DNA may be partially digested with a sufficient amount of an appropriate restriction enzyme to attain complete digestion of the DNA within a specified amount of time (e.g., 1 h). For example, 1-5 units of enzyme may be used per μg of DNA. Partial digestion may be achieved by withdrawing samples of the reaction at various time points, then stopping the reaction (e.g., by adding the sample to a volume of EDTA). Restriction enzymes that are appropriate for partial digestion of genomic DNA include, without limitation, Tsp509 I (NEB), which may be cloned into the Eco RI sites of vectors; Mae II (BMB), Msp I (NEB), and Taq^{α} I (NEB), all of which may be cloned into Cla I sites of vectors; and Sau 3A (NEB), which may be cloned into Bgl II sites of vectors.

Optimal partial digestion of genomic DNA may be determined as a function of limiting time, rather than limiting enzyme, so as to avoid site preferences that exist because of the topological heterogeneity in genomic DNA. In particular, it may be appropriate to determine optimal partial digestion by the length of time needed for the majority of the sample to consist of fragments falling within a particular size range (e.g., from 500 bp to 4 kb). These conditions then may be used to digest a larger sample of chromosomal DNA under identical conditions. DNA samples may be isolated by electrophoresis (e.g., TAE/agarose gel electrophoresis). In a particular embodiment of the present invention, genomic inserts in the size range of 500 bp to 2 kb may be purified (e.g., by extraction with phenol), although it is to be understood that other sizes of fragments may be purified as genomic inserts for use in the present invention.

Once prepared, the genomic inserts of the present invention may be ligated into operable control systems compatible with yeast host cells (e.g., those which are promoters for the synthesis of glycolytic enzymes, such as GAL1), and cloned into suitable cloning vectors which are capable of replication in yeast host cells (e.g., yeast vectors employing the 2-μm origin of replication) (21). Vectors capable of replication include phage vectors and plasmid vectors, such as those known in the art.

Common plasmid vectors include those derived from pBR322 and the pUC series. Charon lambda phage is a commonly employed phage vector. Vectors which may be useful in the method of the present invention include, without limitation, pAS2-1 (Clontech), pGAD424 (Clontech), pB42 (Clontech), pLexA (Clontech), and plex(OP)8-lacZ (Clontech). In the method of the present invention, it may be suitable to use the pB42 cloning vector, which does not express gene fusions in yeast when grown in glucose media. It also may be suitable to use a modified pB42 vector, in which the multiple cloning site (MCS) has been expanded to include additional restriction enzyme recognition sites, as described herein. To limit the loss of plasmids that express important fusions, genomic DNA may be cloned behind the GAL1 promoter, and expressed in glucose, as described herein.

In the method of the present invention, plasmid (cloning vector) DNA may be prepared for ligation with genomic inserts by digestion to completion with a restriction enzyme (e.g., Bgl II, Cla I, Eco RI, Xho I, etc.). The plasmid DNA then may be precipitated (e.g., with ethanol and ammonium acetate) according to standard protocols, and resuspended (e.g., in water, buffer, and shrimp alkaline phosphatase). The plasmid DNA sample then may be heat-inactivated, and the plasmid DNA purified (e.g., by TAE gel electrophoresis and Qiagen QiaQuick clean-up prep).

In the method of the present invention, partially-digested genomic inserts are ligated into an appropriate cloning vector (e.g., one of those described herein) under suitable ligation conditions, to produce ligation products. It may be appropriate to perform this ligation reaction in two parts. First, the optimal vector:insert ratio may be determined empirically. For example, vectors may be incubated with increasing amounts of insert DNA (e.g., from 50 ng to 500 ng) in a ligation reaction of constant volume (e.g., 50 μl) at an appropriate temperature and for an appropriate time (e.g., at 16° C., overnight) using T4 DNA ligase (NEB). Samples for each condition may be transformed into E. coli, and the number of colonies may be scored as a measure of the optimal ratio for the ligation. Once this ratio is determined, new ligation reactions may be set up and used for the transformation in yeast.

The second part of each ligation reaction involves the transformation of the ligation product into a particular yeast strain. Any yeast strain may be appropriate for use in the method of the present invention, including any of those described herein. In particular, it may be suitable to use yeast strain YM4271—a strain that does not contain any bait or reporter constructs, both of which are introduced by mating. The maximal transformation efficiency of the particular yeast strain may be determined systematically, and the library may be transformed directly into yeast by lithium acetate-mediated transformation. For example, competent yeast cells may be grown to a particular OD (e.g., 0.8, at A_{600 mn}), incubated in LiOAc at an appropriate temperature and for an appropriate time (e.g., at 4° C., overnight), and administered heat shock at an appropriate temperature and for an appropriate time (e.g., at 42° C., for 30 min) in a transformation mixture (e.g., a mixture containing DMSO). The cells then may be incubated with the ligation mixture in the transformation mixture.

Following heat shock, the cells may be centrifuged at an appropriate speed, for an appropriate time (e.g., at 5K rpm in a microfuge, for 3 min). To prepare for screening, the cells may be resuspended (e.g., in YPD) at an appropriate temperature, for an appropriate time (e.g., at room temperature, for 2 h). The cells then may be pooled and added to SD-Trp. The cells may be grown with moderate agitation for a suitable amount of time (e.g., 16 h). The culture then may be chilled, pelleted and resuspended in an appropriate medium (e.g., glycerol), and frozen at a suitable temperature (e.g., 70° C.).

The expression library of the present invention is not introduced into a prokaryotic host at any stage during its preparation; rather, the library is prepared in vitro, and then propagated/amplified directly in yeast. Thus, this expression library differs from other bacterial genomic libraries because it is constructed without passage through E. coli at any step. The logic behind this modification is based on the observations that many E. coli genes are toxic when expressed on plasmids, and that the passage and amplification steps of library construction will cause a severe underrepresentation of such plasmids. The inventors have shown that such underrepresentation of toxic genes can be limited by forgoing the use of E. coli as a host, and by keeping the expression of such genes repressed in yeast.

By transforming ligation products directly into yeast, without passaging through a prokaryotic host, the present inventors have enabled a method that permits, for the first time, the construction of an expression library of bacterial genomic DNA, having increased complexity, that is stored in a yeast host strain. This expression library represents an improvement over other prokaryotic genomic libraries currently available. Because amplification and transformation take place directly in yeast, rather than in E. coli, the complexity of the inventors' expression library is increased. In particular, loss of functional clones that may be toxic to E. coli or other bacteria—a frequent occurrence when gene fusions are inappropriately expressed during the passaging phase—is avoided. Thus, the expression library of the present invention may be suitable for use in a two-hybrid method.

It is expected that the inventors' expression library will permit the cloning of genes toxic to E. coli, B. subtilis, or other bacteria, which often go undetected when screening expression libraries that have been passaged through a prokaryotic host, such as E. coli, at any stage of preparation. Such toxic genes represent potential new targets for the development of antimicrobial compounds. Accordingly, the present invention further provides a method for screening for a gene that is toxic to bacteria, particularly a gene that is toxic to E. coli, B. subtilis, or another bacterium when inappropriately expressed (e.g., from a gene fusion). Such a toxic gene may be involved in important cellular processes in bacteria (e.g., cell division, transcription, or translation).

The method of the present invention comprises the following steps: (a) obtaining an expression library made in accordance with the method of the present invention described above; and (b) screening the expression library for a gene that is toxic to bacteria. In screening an expression library such as that of the present invention, the objective is to recover as many plasmids as possible, including those containing toxic inserts. The expression library described herein is useful, in that it preserves many toxic inserts that otherwise would be lost if the plasmids were transformed in bacteria, in addition to the plasmids that are more easily recovered.

The expression library of the present invention may be screened for genes, including genes that are toxic to bacteria, using any one of several different methods known in the art. For example, the expression library of the present invention may be used to screen for genes using the Y2H method. The expression library also may be screened using a yeast mating-selection strategy, as previously described (22). In a particular embodiment of the present invention, the expression library is screened using a mating strategy.

Using a simple yeast two-hybrid system, for example, new interacting proteins that are toxic to bacteria may be screened by co-transforming a yeast strain containing an integrated copy of a reporter gene (e.g., UAS_{GAL1}-lacZ) with two plasmids, as previously described (5). One plasmid may encode a fusion of the DNA-binding domain of the yeast transcription factor, Gal4, to a protein of interest (e.g., a protein, such as E. coli ZipA, that plays a role in controlling cell division, transcription, translation, or another important cellular process in bacteria). The other plasmid (the library plasmid) may encode a fusion of the transcriptional activation domain of Gal4 to randomly-generated coding regions obtained from the expression library of the present invention. If a protein encoded by the library fusion plasmid interacts with the protein of interest, the transcription activation domain of Gal4 becomes co-localized upstream of the reporter gene, and transcription is activated (5).

Alternatively, a yeast mating-selection scheme may be used to detect new protein-protein interactions, and to identify new genes that are toxic to bacteria, in accordance with methods previously described (22). Using this strategy, two plasmids (e.g., a plasmid containing the expression library of the present invention and a plasmid encoding a protein of interest, as described above) may be brought together in a single cell by mating, rather than by transformation. Diploids containing interacting fusion proteins then may be selected using a reporter construct (e.g., UAS_{GAL1}-HIS3 or UAS_{GAL1}-LEU2) that has been integrated into the yeast genome. The specificity of the interaction may be confirmed by testing the ability of the selected clones to activate the expression of the lacZ reporter gene that also has been integrated into the genome (22).

To screen the expression library of the present invention using a mate-selection (mating) strategy, frozen aliquots of the expression library contained in a yeast host may be revived by thawing on ice, then incubated in YPD at an appropriate temperature and for an appropriate time (e.g., at 30° C., for 2 h, with mild agitation). The revived library then may be mixed with a yeast culture containing a fusion with the protein of interest (e.g., a culture of EGY48pLexA-ZipA). The culture may be allowed to mix for a suitable length of time (e.g., 6 h), to permit zygotes to form. Diploids then may be plated onto SD-LUTH/GalRaf plates (23), and incubated at an appropriate temperature and for an appropriate time (e.g., at 30° C., for 5 days). Titering data may indicate the number of diploids that are plated. The plates also may be replica printed onto SD-LUTH/GalRaf/X-Gal plates (23).

Plasmid DNA may be obtained and used to transform a bacterial host (e.g., E. coli strain KC8), such that transformants contain any of three plasmids expressed in yeast (the bait, the prey, or the reporter). For example, all diploids containing prey plasmids that putatively interact with the protein of interest (e.g., ZipA) may be inoculated into a ⁻Trp medium and grown overnight. The cultures may be pelleted and resuspended in an appropriate solution (e.g., solution P1 from a Qiagen Turbo Miniprep kit that has been supplemented with Zymolyase (ICN Biologicals)). The cultures then may be incubated at an appropriate temperature and for an appropriate time (e.g., at 37° C., for 30 min). Further steps in the purification of plasmid DNA using the Miniprep kit then may be performed as per the manufacturer's instructions. The eluted DNA miniprep may be used to transform the bacterial host by electroporation. After transformation, prey plasmids then may be selected. For example, transformants may be plated onto M9 minimal plates supplemented with leucine and uracil, to permit selection of only those cells that are transformed with the prey plasmid.

For transformants that are stable in E. coli, new transformations in yeast may be made from DNA purified from single colonies. These transformants then may be assayed for the specificity of their interaction with a protein of interest. Prey plasmids that appear specific for the protein of interest may be analyzed by sequencing their inserts. From the classes of prey, a list of putative interactors then may be compiled.

Accordingly, using a Y2H method, a mating-selection strategy, or another method of screening for genes, it may be possible to identify a protein that plays a role in controlling cell division, transcription, translation, or another important cellular process in a bacterium. Inhibition of, or interference with, such a protein could interfere with cell division, transcription, translation, or another important cellular mechanism in a bacterium, and thereby prevent the growth or proliferation of the bacterium. Thus, it is expected that an inhibitor of such a protein would represent a potential new target for the development of antimicrobial compounds.

Moreover, using a Y2H method, a mating-selection strategy, or another method of screening for genes, it may be possible to identify a protein that interacts with a second protein that plays a role in controlling cell division, transcription, translation, or another important cellular process in a bacterium. Through its interaction with a second protein that controls an important cellular process in a bacterium, such a protein interactor could interfere with cell division, transcription, translation, or another important cellular mechanism, and thereby prevent the growth or proliferation of the bacterium. Thus, it is expected that such a protein interactor would be encoded by a gene that is toxic to bacteria, and would represent a potential new target for the development of antimicrobial compounds.

To identify a gene which codes for a protein interactor, genomic inserts may be isolated, then subjected to sequence analysis or identified by BLAST (2). Samples from an amplified expression library may be streaked onto SD-Trp plates, for example, and grown as single colonies for an appropriate time (e.g., as 5-ml overnight cultures); the plasmids then may be isolated as described (23). Once isolated, the plasmids may be analyzed by transformation into an appropriate bacterial strain (e.g., E. coli strain KC8) by electroporation, as described (23). Thereafter, the gene products may be sequenced, and the genomic inserts may be identified by BLAST (2).

Plasmids that are easily recovered (e.g., those that do not contain toxic inserts), as well as plasmids that contain genes that are toxic to bacteria (e.g., E. coli), may be analyzed by transformation into bacteria. For example, a toxic insert that causes a 10-fold reduction in growth rate may be represented once at the time of transformation in E. coli, if that method is used for amplifying the expression library, and 0.1 times, if the pool of transformants is grown for 3.2 doublings (thereby permitting a 10-fold increase in cell number). For log-phase E. coli, for example, this occurs in a little more than 1 h. If the culture is amplified for 5-8 h, that insert may represent a very small number of plasmids in the final library. Nevertheless, a 10-fold reduction in growth rate still may result in the formation of a colony; the colony just would appear later than (e.g., 1 day later than) the colony formed by a strain carrying a non-toxic plasmid. Accordingly, following transformation into bacteria, a toxic gene still may be recovered and sequenced.

Where gene inserts are too toxic to be recovered in E. coli or another bacterium, the toxic inserts may be identified by PCR of a yeast plasmid miniprep (e.g. using Taq polymerase), followed by sequence analysis and identification by BLAST (2). PCR and sequence analysis may be followed by one of several methods (e.g., cloning of an insert under a particularly repressed promoter, such as AraB, or alteration of growth conditions to favor the growth of the clone in E. coli).

The sequences of inserts from the expression library of the present invention may be used to determine if any of the clones represents a potentially interesting interaction. Searches of GenBank (Los Alamos, N. Mex.) and EMBL (Heidelberg, Germany) data bases may be conducted in order to identify known sequences such as OrfE—a previously characterized protein that interacts with ZipA, and which was identified by the inventors using the expression library of the present invention. A number of methods then may be used to characterize the novel genes that encode these protein interactors, and to begin to determine their functional roles in such important cellular processes as cell division, transcription, and translation in bacteria. In particular, the identified genes may be searched against the literature to determine what is known about them. Additional methods also may be used to categorize the genes screened by the above-described methods. For example, gene fragments that are toxic when expressed as fusions to eukaryotic activation domains are prime candidates for genes (or homologues of genes) that require a high level of control over their expression in E. coli. Such genes are obvious targets for intervention with chemotherapeutics.

Once a gene toxic to bacteria has been screened using the expression library and screening methods of the present invention, and has been determined to have suitable binding affinity to a gene (e.g., ZipA) involved in an important cellular process in bacteria, such as cell division, transcription, or translation, it may be evaluated to ascertain whether it has an effect on bacterial proliferation. In particular, the gene product may be assessed for its ability to act as an inhibitor to cell division, or to otherwise function as an appropriate antimicrobial agent. Protein-protein interactions identified using the expression library and screening method of the present invention may be used as tools in the development of drug screens, as targets for small-molecule inhibitors that can act as antimicrobial agents, and as a basis for peptidomimetics. Such drugs, inhibitors, and peptidomimetics may be useful for treating a subject infected with a bacterium, by administering to the subject an effective amount of the drug, inhibitor, or peptidomimetic.

The present invention is described in the following example, which is set forth to aid in the understanding of the invention, and should not be construed to limit in any way the scope of the invention as defined in the claims which follow thereafter.

### EXAMPLE

1. Introduction

The present example was conceived out of the inventors' theory that toxicity is a problem for two-hybrid screening with a prokaryotic library. The inventors had observed that screens to identify genes that interact with FtsZ—the E. coli cell-division gene—failed to identify genes that were known to interact with FtsZ. These negative results were correlated with the knowledge that FtsZ, and many other genes encoding components of the cell-division machinery, are unstable when cloned on plasmids, even if the promoter element is removed. Accordingly, the inventors decided to develop their own library. This provided them with the opportunity to observe all aspects of the generation of a two-hybrid library, and to determine the means by which the construction of such a library can best preserve complexity for a prokaryotic genome.

2. Materials and Methods

A. Media and Strains

Yeast strains EGY48 and YM4271, E. coli strain KC8, and plasmids pLexA, plex(OP)8-lacZ, and pB42, were obtained from Clontech, and were described previously (19). E. coli strain DM1 was obtained from GibCo. Media were obtained from Difco and Bio101, and were prepared according to standard recipes (1, 9).

B. Preparation of E. coli Chromosomal DNA

A cell culture of 100 ml of E. coli strain MG1655, grown in 2xYT, was pelleted and washed with 50 ml of TES buffer (50 mM Tris-HCl, pH 8.0; 5 mM EDTA; and 50 mM NaCl). The cells were pelleted again, resuspended in 24 ml of 25% sucrose and 50 mM Tris (pH 8.0), and incubated on ice for 20 min. 12 ml of a solution containing 10 mg/ml lysozyme and 0.25 mM EDTA (pH 8.0) was added, and the solution was mixed by inversion several times. 7.5 ml of a lysis solution (5% sarkosyl; 50 mM Tris, pH 8.0; and 62.5 mM EDTA) was added, and the sample was mixed by inversion. 5 ml of a 10 mg/ml solution of proteinase K was added. The sample was mixed by inversion, then incubated at 55° C. for 60 min. The sample was extracted with an equal volume of TE-saturated phenol (TE: 10 mM Tris-HCl, pH 8.0; 1 mM EDTA, pH 8.0), and again with PCI (TE-saturated phenol, chloroform, and isoamyl alcohol; 25:24:1). 2-3 mg of DNA were typically recovered.

C. Preparation of B. subtilis chromosomal DNA

100 ml of B. subtilis strain 168 were grown to saturation in BHI (GibCo) for 16 h. The cells were washed once in 50 ml of EET (100 mM EDTA, 10 mM EGTA, and 10 mM Tris; pH 8.0). The cells then were resuspended in 15 ml of EET. 1.5 ml of a 10 mg/ml solution of lysozyme and 0.5 ml of a 10 mg/ml solution of RNase A were added. The solution then was incubated for 30 min at 37° C. An additional 4.5 ml of EET were added, along with 1.5 ml of 20% SDS and 1 ml of a 10 mg/ml solution of proteinase K. The solution was incubated for 10 min at 65° C. 1 ml of 5 M NaCl was added, and the solution was mixed thoroughly. 0.8 ml of a 10% CTAB/0.7 M NaCl solution were added, and the solution was mixed thoroughly again. The solution was extracted with 20 ml of PCI, and the aqueous phase was transferred to a new tube. 4.5 ml of isopropanol were added, and the solution was incubated for 15 min at room temperature, then centrifuged at 15K rpm for 20 min. The pellet was washed with 70% ethanol, recentrifuged, and resuspended in 1 ml of TE (10 mM Tris-HCl, pH 8.0; 1 mM EDTA, pH 8.0).

D. Construction of Modified pB42 Vectors, and Preparation for Ligation with Genomic Inserts

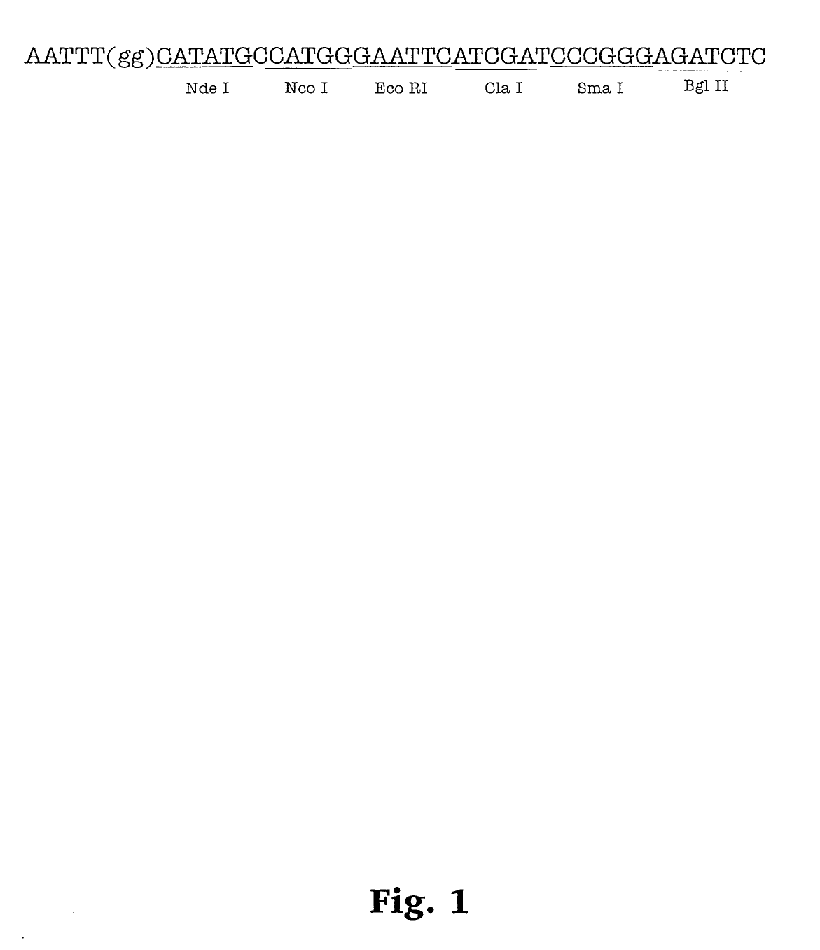

Plasmid pB42 was digested to completion with Xho I and Eco RI, then precipitated and digested with CIP. This vector then was split into three reactions, in which three pairs of oligos were added to form the new multiple cloning sites. The oligo sequences are shown in FIG. 1. After annealing, the reactions were incubated overnight with T4 DNA ligase, and transformed into E. coli. The three plasmids to be digested with Cla I were transformed into E. coli strain DM1, and transformants were prepared using the Qiagen Plasmid Maxiprep kit, according to the manufacturer's instructions. For each set of transformation reactions, plasmid DNA was prepared as follows: 100 μg of DNA were digested to completion with Eco RI, Cla I, or Bgl II. DNA was precipitated with ethanol and ammonium acetate, according to standard protocols, and resuspended in 50 μl of water, buffer, and shrimp alkaline phosphatase, as described by the manufacturer (BMB). The sample was heat inactivated, and the DNA was purified by TAE gel electrophoresis and Qiagen QiaQuick clean-up prep. 100 ng of DNA were transformed into yeast strain YM4271, as described below, to ensure that very little closed circular DNA (ccDNA) remained, as measured by the number of colonies resulting from such a transformation. The number of transformants measured by such a test ranged from 0 to 60. In contrast, 100 ng of ccDNA typically yields 10⁴ colonies or greater, under similar transformation conditions.

E. Preparation of Genomic Inserts

Chromosomal DNA from E. coli strain MG1655 was digested with a sufficient amount of the indicated restriction enzyme to attain complete digestion of the DNA within 1 h (typically, 1-5 units of enzyme/μg of DNA). Partial digestion was achieved by withdrawing samples of the reaction at various time points, and stopping the reaction by adding the sample to an equal volume of 20 mM EDTA on ice. The following enzymes were used for this procedure: Tsp509 I (NEB), which was to be cloned into the Eco RI sites of the vectors; Mae II (BMB), Msp I (NEB), and Taq^{α} I (NEB), all of which were to be cloned into the Cla I sites; and Sau 3A (NEB), which was to be cloned into the Bgl II site. Optimal partial digestion of the DNA was determined by the length of time needed for the majority of the sample to consist of fragments that ranged in size from 500 bp to 4 kb. These conditions, including an optimal digestion time that was typically 2-5 min, then were used to digest a larger sample of chromosomal DNA under identical conditions. These samples were isolated by TAE/agarose gel electrophoresis. Samples in the size range of 500 bp to 2 kb were purified by extraction with phenol. The same steps also were performed using chromosomal DNA from B. subtilis. The transformants resulting from B. subtilis chromosomal DNA are summarized in Table 1.

TABLE 1
Transformants produced from B. subtilis chromosomal DNA

| Pool | Vector digest | Insert digest | Transformants |
|---|---|---|---|
| B1 | Eco RI | Tsp509 I | 998,000 |
| B2 | Cla I | Msp I | 165,000 |
| B3 | Cla I | Mae II | 260,000 |
| B4 | Cla I | Taq^{α} I | 220,000 |
| B5 | Bgl II | Sau 3A | 1,220,000 |

F. Ligation and Transformation in Yeast

Ligation reactions for each set of partially-digested inserts were performed in two parts. In the first, the optimal vector:insert ratio was determined empirically. 100 ng of vector were incubated with increasing amounts of insert DNA (typically, from 50 ng to 500 ng) in a ligation reaction of constant volume (50 μl) overnight, at 16° C., using T4 DNA ligase (NEB). Samples for each condition were transformed into E. coli, and the number of colonies was scored as a measure of the optimal ratio for the ligation. When this ratio was determined, new ligation reactions were set up (four reactions, 500 μl each) and used for the transformation in yeast.

The second part of each reaction involved the transformation of the ligation into yeast. The maximal transformation efficiency of yeast strain YM4271 was determined systematically. For this strain, maximal efficiency was achieved after preparing the competent cells that had been grown to an OD (A_{600 nm}) of 0.8, incubating the cells in 0.1 M LiOAc overnight at 4° C., and administering heat shock at 42° C. for 30 min in a transformation mixture that contained 10% DMSO. For the transformation of the ligation products into yeast, 40 transformation tubes were set up. In each, 50 μl of cells were incubated with 50 μl of the ligation mixture in the transformation mixture. Following heat shock, the cells were centrifuged at 5K rpm in a microfuge for 3 min. The cells were gently resuspended in 100 μl of YPD, and allowed to sit at room temperature for 2 h. The cells then were pooled and added to 500 ml of SD-Trp. 100 μl of the mixture were removed for titering, and the remaining culture was grown with moderate agitation for 16 h. The cultures then were chilled, pelleted and resuspended in 50 ml of 15% glycerol, and frozen at ⁻70° C. The samples removed for titering were serially diluted and plated onto ⁻Trp plates for determination of the total number of transformants. A sample of the frozen stock was also titered.

G. Characterization of the Library

Identification of inserts toxic to E. coli were characterized as follows. Samples from the amplified library pools were streaked onto SD-Trp plates, and grown as single colonies. Colonies were grown as 5-ml overnight cultures, and the plasmids were isolated as described (23). Plasmids were either analyzed by transformation into the bacterial strain KC8 by electroporation, as described (23), or amplified by PCR, from the yeast plasmid miniprep, using Taq polymerase and oligos that flank the MCS (5′-CTCTGGC GAAGAAGTCCAAAGCTTCTCG, 5′-CAGCCTGACTGGCTGAAATCGAATGGTTT TC). The products were sequenced, and the genomic inserts were identified by BLAST (2).

3. Results

A. Advantages of a new two-hybrid library

The inventors examined the steps involved in making a two-hybrid library, attempting to identify those steps that were particularly important for maintaining the complexity of a prokaryotic genomic library. The most important factor was the avoidance of passaging the library through E. coli at any stage. However, to accomplish such a step, ligation products generally have to be transformed directly into yeast. This makes the generation of the library much more difficult, since the transformation efficiency of yeast is much lower than that of E. coli. If the library is intended for use in more than one screen, an additional element must be introduced: the library has to be transformed into a host strain that does not contain any bait plasmid. The introduction of a bait plasmid for each screen is accomplished by mating.

The inventors ultimately selected yeast strain YM4271 for use in the present invention because it performs well in mating-based two-hybrid assays using the Lex system. For this strain, however, a reporter has to be introduced by mating. Because the strain lacks both bait and reporter constructs, there is significant flexibility in terms of these plasmids: improvements in either the bait or the reporter can be incorporated into later screen designs, as described below.

Observations made by the inventors while working with FtsZ in a two-hybrid system also influenced their decision to develop a library in a pB42-based plasmid. In particular, it appeared to the inventors that FtsZ was somewhat toxic in yeast and E. coli. FtsZ hybrids in the Gal system showed a reduction in growth rate, and cells appeared abnormal when viewed by phase-contrast microscopy (results not shown). The reduction in growth rate was most severe in the Lex system, when FtsZ was expressed as a fusion to the B42 activation domain. This fusion was under the control of the GAL1 promoter, and was not expressed in glucose. Growth in galactose greatly exacerbated the slow-growth phenotype, suggesting that the expression of FtsZ was toxic to yeast. As Table 2 shows, the expression of the B42-FtsZ fusion had a dramatic effect on the stability of the plasmid.

TABLE 2
Plasmid loss during growth in rich media

| Plasmid | Medium | Plate | Colonies | Percent lost |
|---|---|---|---|---|
| pB42 | YPD | YPD | 112 | 13 |
| | | SD-Trp | 97 | |
| | YPGal | YPD | 127 | 13 |
| | | SD-Trp | 110 | |
| pB42-FtsZ | YPD | YPD | 126 | 8 |
| | | SD-Trp | 116 | |
| | YPGaL | YPD | 98 | 92 |
| | | SD-Trp | 8 | |

In the present example, yeast strain YM4271 was transformed with plasmids pB42 and pB42-FtsZ. These transformants were mated with strain EGY48, which had been transformed with plasmid pLexA. Overnight cultures of the resulting diploids (grown in SD-Trp medium) were used to inoculate 100 ml each of YPD and YPGal media. Cultures were grown for 5 h; aliquots then were removed and serially diluted onto YPD plates and SD-Trp plates. Individual colonies were counted and tabulated. The results of one experiment are presented in Table 2 (above).

As Table 2 shows, expression of the pB42-FtsZ fusion, through growth in galactose-containing medium, is highly toxic to yeast. The effect was not as dramatic when the Gal4AD-FtsZ fusion was compared with a vector control grown in YPD for several generations (results not shown). However, even subtle differences in the growth rate of functional clones, through the constitutive expression of plasmids in the Gal system, would be an additional liability for maximum complexity of the library. Thus, the inventors believed that a library cloned behind the GAL1 promoter, and expressed in glucose, offered the best means of limiting the loss of plasmids that express important fusions.

The inventors decided to base their strategy for cloning on that developed by James et al. (12). As reported, James et al. modified the multiple cloning site of pGAD424 to allow the ligation of insert digested with a family of restriction enzymes that cleave DNA at various four-base recognition sequences, thereby producing two-base overhangs that can be cloned into a Cla I restriction site. The inventors developed polylinkers that included a Cla I site and other restriction sites, such as Eco RI and Bgl II. The latter two restriction sites allow the cloning of DNA that has been digested with Tsp509 I and Sau 3A, respectively.

Enzymes Tsp509 I and Sau 3A were included in the example because of the increased efficiency of ligation of restriction fragments with four-base-pair overhangs, as compared with that of the two-base-pair overhangs generated by enzymes that allow cloning into the Cla I restriction enzyme site. Increased ligation efficiency was considered to be a factor to compensate for the lower efficiency of transformation into yeast, relative to E. coli. The multiple cloning site cassette was constructed in all three reading frames. DNA from E. coli strain MG1655 was chosen for the first library because it is robust for growth, and because it was the strain chosen for genomic sequencing. Identification of the inserts after recovery from a screen is relatively easy, because MG1655 is a very well-characterized strain of E. coli. Chromosomal DNA was isolated and prepared for cloning by partial digestion with the enzymes listed in Table 3.

TABLE 3
Primary transformants in yeast for each pool

| | | | | | | | | Expected |
|---|---|---|---|---|---|---|---|---|
| | | | Site | Average | Predicted | | | # of 5′ |
| | Vector | Insert | frequency | fragment | number of | Trans- | | ends |
| Pool | digest | digest | (%) | size (bp) | 5′ ends | formants | F₁ | missing |
| E1 | Eco RI | Tsp5O9 I | 0.39 | 257 | 17864 | 1,560,000 | 1.1845E-38 | 0 |
| E2 | Cla I | Msp I | 0.60 | 167 | 27600 | 240,000 | 0.00016729 | 4.6 |
| E3 | Cla I | Mae II | 0.33 | 303 | 15180 | 230,000 | 2.6276E-07 | 0 |
| E4 | Cla I | Taq^{α} I | 0.39 | 257 | 17864 | 230,000 | 2.5602E-06 | 0 |
| E5 | Bgl II | Sau 3A | 0.39 | 257 | 17864 | 1,310,000 | 1.4174E-32 | 0 |

In the present example, DNA ligations were transformed directly into yeast strain YM4271. To ensure that transformation was performed as effectively as possible, the strain was carefully characterized to determine the conditions under which it had the highest transformation efficiency. Yeast strains are readily transformable by both electroporation and lithium acetate (3, 14). However, the conditions under which they are transformed with maximum efficiency are strain-dependent. For example, in the lithium acetate transformation procedure, some strains transform well after a 15-minute heat shock, while others do not transform well with less than a 30-minute heat shock (7, 14).

As described above, purified partial digests were mixed with digested, phosphatased vector DNA in titration experiments, then scored by transformation in E. coli, in order to determine the number of successful ligations. The amount of vector DNA and the ratio of insert-to-vector DNA were examined. Optimal conditions determined in the test reactions were repeated in multiple ligations for each sample of insert DNA. Ligation reactions were transformed into yeast that had been made competent by treatment with lithium acetate. Transformation reactions were pooled by insert preparation, but not by vector reading frame, and used to inoculate 500 ml of ⁻¹Trp media. Cultures were grown to an OD(A_{600 nm}) of 0.8 to 1.2, pelleted, and resuspended in 50 ml of medium for freezing. Aliquots were taken after transformation, to determine the total number of transformants, and after freezing, to determine the titer. The results are presented in Table 3 (above).

Table 3 lists the restriction enzymes used to generate the fragments, their cut-site frequencies, and the total number of fragments generated for a complete digestion (or total 5′ ends). The table also includes an estimate of the extent to which all potential clones have been generated, as described by Finkel et al. (6). Because of the relatively small size of the E. coli genome, it is easy to obtain good coverage of the genome using the total number of primary transformants obtained by the inventors. The low F₁ values in Table 3 reflect the occurrence of pooling during the transformation in yeast. The actual number of missing clones is higher, because the functionality of any given fusion is dependent upon the reading frame; therefore, inclusion in the library requires cloning in all three reading-frame vectors, not just one vector. While cloning directly in yeast does increase the probability of missing particular 5′ ends, the increased probability is not unacceptably high.

B. Analysis of the Library

The first step in the analysis of the expression library is the determination of the fraction of clones that contain inserts. The second step in the analysis consists of the identification of inserts that are stable in yeast, but not in E. coli. Transformants from the yeast library pools were isolated as single colonies. Their plasmids also were isolated, then transformed into E. coli. In respect of the colonies recovered by transformation into E. coli strain DH10B (GibCo), a majority of the plasmids characterized by restriction digest showed inserts.

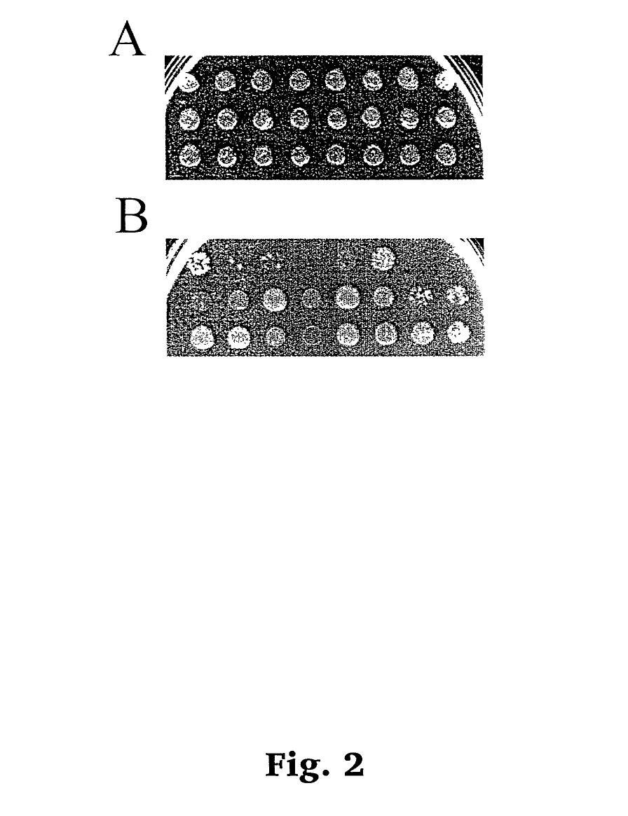

The inventors tested the rationale that cloning directly in yeast permits the inclusion of unstable clones, particularly those that encode fusions that are growth-inhibitory due to inappropriate expression in E. coli. In order to characterize the stability of the plasmids in E. coli, diploids of the library-containing yeast strain YM4271 were mated with strain EGY48, which contained the pLexA vector and the plex(OP)8-lacZ reporter construct. From these diploids, plasmids were recovered and transformed into E. coli strain KC8, allowing the specific rescue of bait plasmids through complementation of the trpB mutation by the TRP1 gene. Following transformation, some transformants were spotted onto plates that allowed for the selection of any of the plasmids (vector, reporter, or library), because each plasmid conferred ampicillin resistance (FIG. 2A). Other transformants were spotted onto plates that specifically selected for the library plasmid, because it is the only plasmid that complements the trpB mutation of the host (FIG. 2B).

All of the transformants shown in FIG. 2A grew as spots. In contrast, several of the transformants shown in FIG. 2B did not grow. In particular, while some of the spots represented in FIG. 2B showed meager growth after 16 h at 37° C., others did not grow at all (cf. the first, second, and fourth spots in the second row in FIG. 2B). For those that did not show any growth, it is possible that mutations in the E. coli portions of the shuttle plasmids were corrupted, such that they could no longer support growth of E. coli in the presence of ampicillin. However, for those that were growth-retarded, the inventors sought to determine whether the strain carried a hybrid that could account for the aberrant growth. Accordingly, several plasmid inserts were amplified by PCR and sequenced.

The inserts were in-frame fusions of the B42 activation domain and the genomic sequences listed in Table 4. From the list of inserts, the inventors found that membrane proteins appeared to be the major class of fusions that inhibited growth in E. coli. While membrane proteins have been cloned successfully from two-hybrid libraries (18), they may be difficult to work with in a two-hybrid system (4, 13). The important distinction to note here is that many fusions may be lost or underrepresented in a two-hybrid library, due to growth defects caused by inappropriate expression in E. coli. Additional fusions that may be lost for similar reasons include genes that control important cellular processes in bacteria, such as cell division (as described above), transcription, and translation (Table 4).

TABLE 4
Fusions to B42 that are unstable in E. coli

| Insert | Comments |
|---|---|
| YdaW | putative membrane protein |
| YaaU | probable transporter |
| PhoQ | transmembrane receptor |
| YjcE | putative Na⁺/H⁺antiporter |
| GalR | gal operon repressor protein |
| YicE | hypothetical xanthine/uracil permease |
| PrfC | polypeptide release factor 3 |

C. Design of the Library Screen

The library can be screened with a mating strategy. For example, the library in strain YM4271 can be revived and mixed with an excess of strain EGY48 that contains the bait plasmid (pLexA) and the reporter constructs (the high-copy plasmid pLexA(OP)8-lacZ and the chromosomal LexA(OP)6-LEU2 fusions). The strains are incubated in YEPD for 2-4 h, allowing zygotes to form, but not permitting resumption of significant growth. Diploids that carry presumptive interacting proteins then can be selected on LUTH/GalRaf plates. These are screened directly by replica printing onto LUTH/Xgal/GalRaf plates.

Colonies can be miniprepped to recover the prey plasmids from strains that score positive. Prey plasmids that are recovered as stable transformants in E. coli may be retested by retransforming them into yeast, and rechecking the Leu+and X-Gal+phenotypes, along with controls that show whether these phenotypes are specific for the bait interaction. Plasmids that cannot be recovered in E. coli can be analyzed by PCR and sequence analysis of the insert. The sequences of the inserts can be used to determine if any of the clones represents a potentially interesting interaction.

PCR and sequence analysis can be followed by one of several methods, including cloning of the insert under a particularly repressed promoter, such as AraB, or alteration of growth conditions to favor the growth of the clone in E. coli. For example, many of the genes that are required for cell division exhibit inappropriate expression in a two-hybrid system. However, they have reduced toxicity when co-expressed with pZAQ, a plasmid that expresses FtsZ, FtsA, and FtsQ in amounts and proportions that allow cell division to continue.

4. Conclusion

The strategy described herein seeks to address several factors that may limit the complexity of genomic libraries, particularly two-hybrid libraries. Two-hybrid libraries, and some other expression libraries, have unusually high requirements for the number of clones needed to ensure complete representation of a genome. The one factor that most improves this strategy is transformation of the ligation products directly into yeast, rather than the more usual E. coli host. Transformation directly into yeast has strong advantages and disadvantages. One advantage is that this method directly addresses the potential of clones to be lost from the library during the amplification and passaging stages. Toxicity, as a consequence of expression in yeast, also may be limited by maintaining the library in a transcriptionally-dormant state through growth in glucose. Gene fragments that are toxic when expressed in E. coli are particularly troubling in a search for antibacterial targets, because they represent exactly those genes of greatest interest, i.e., genes where expression is tightly controlled because they impinge on very sensitive processes.

A strong disadvantage of the development of the inventors' library in yeast is the significantly lower transformation efficiency of yeast, as compared with E. coli. Using this method, significantly more effort is required to create a library with a theoretical coverage of the E. coli genome than is required using more traditional methods of library construction and amplification. As such, transformation in yeast is not likely to replace E. coli as a standard method of library construction. However, it should still have a role in the development of a complete genomic analysis when there is reason to suspect that toxicity is a concern, as in the circumstances described herein.

The two-hybrid system is constantly evolving. One advantage of the library design described herein is that it allows the incorporation into later screens of improvements in reporter and bait constructs. For example, the use of a lexA(OP)-HIS3 reporter has facilitated some of the technical aspects of two-hybrid screens (16, 17). Different DNA binding proteins, such as cI and the lac repressor gene products, along with their respective operators and reporters, may reduce or alter the false positives that arise during the screens (8, 11, 15). Both types of changes can be introduced in the strain to be mated to the library strain.

### Cited References

(1) Adams et al., Methods in Yeast Genetics (Cold Spring Harbor: Cold Spring Harbor, Press, 1997).

(2) Altschul et al., Gapped BLAST and PSI-BLAST: a new generation of protein database search programs. Nucleic Acids Res., 25:3389-402, 1997.

(3) Becker and Guarente, High efficiency transformation of yeast by electroporation. In Guide to Yeast Genetics and Molecular Biology, vol. 194, C. Gutherie and G. R. Fink, eds. (San Diego: Academic Press, 1991) 182-87.

(4) Brachmann and Boeke, Tag games in yeast: the two hybrid system and beyond. Current Opinion in Biotechnology, 8:561-68, 1977.

(5) Fields and Song, A novel genetic system to detect protein-protein interactions. Nature, 340:245-46, 1989.

(6) Finkel et al., Determining the probability of obtaining a desired clone in an amplified or shuttle library. BioTechniques, 16:580-82, 1994.

(7) Gietz et al., Studies on the transformation of intact yeast cells by the LiAc/SS-DNA/PEG procedure. Yeast, 11(4):355-60, 1995.

(8) Grossel et al., A yeast two-hybrid system for discerning differential interactions using multiple baits. Nature Biotechnology, 17:1232-33, 1999.

(9) Gutherie and Fink, Guide to Yeast Genetics and Molecular Biology, vol. 194 (San Diego: Academic Press, 1991).

(10) Gyuris et al., Cdk1, a human G1 and S phase protein phosphatase that associates with Cdk2. Cell, 75:791-803, 1993.

(11) Inouye et al., Mutational analysis of STE5 in the yeast Saccharomyces cerevisiae: application of a differential interaction trap assay for examining protein-protein interactions. Genetics, 147:479-92, 1997.

(12) James et al., Genomic libraries and a host strain designed for highly efficient two-hybrid selection in yeast. Genetics, 144:1425-36, 1996.

(13) Johnsson and Varshavsky, Split ubiquitin as a sensor of protein interactions in vivo. Proc. Natl Acad. Sci. USA, 91:10340-44, 1994.

(14) Schiestl and Gietz, High efficiency transformation of intact yeast cells using single stranded nucleic acids as a carrier. Current Genetics, 16(5-6):339-46, 1989.

(15) Serebriiskii et al., A two-hybrid dual bait system to discriminate specificity of protein interactions. J. Biol. Chem., 274(24):17080-987, 1999.

(16) Vojtek et al, Searching for interacting proteins with the yeast two-hybrid system II. In The yeast two-hybrid system, P. L. Bartel and S. Fields, eds.

(Oxford, UK: Oxford University Press, 1997) 29-42.

(17) Vojtek et al., Mammalian Ras interacts directly with the serine/threonine kinase Raf. Cell, 74:205-14, 1993.

(18) Xue et al., GPR1 encodes a putative G protein-coupled receptor that associates with the Gpa2p G-alpha subunit and functions in a Ras-independent pathway. EMBO J., 17:1996-2007, 1998.

(19) Golemis and Khazak, Alternative yeast two-hybrid systems. The interaction trap and interaction mating. Methods Mol. Biol., 63:197-218, 1997.

(20) Beers and Berkow, eds., The Merck Manual of Diagnosis and Therapy, 17^{th} ed. (Whitehouse Station, NJ: Merck Research Laboratories, 1999) 1088-92, 1101-03.

(21) Broach, J. R., Construction of high copy yeast vectors using 2-microns circle sequences. Methods Enzymol., 101:307-25, 1983.

(22) Bendixen et al., A yeast mating-selection scheme for detection of protein-protein interactions. Nucl. Acids Res., 22:1778-79, 1994.

(23) Golemis and Brent, Searching for interacting proteins with the two-hybrid system III. In The Yeast Two-Hybrid System, P. A. Bartel and S. Fields (eds.) (Oxford, UK: Oxford University Press, 1997).

All publications mentioned hereinabove are hereby incorporated in their entireties. While the foregoing invention has been described in some detail for purposes of clarity and understanding, it will be appreciated by one skilled in the art, from a reading of the disclosure, that various changes in form and detail can be made without departing from the true scope of the invention in the appended claims.

What is claimed is:

1. A method for constructing an expression library containing a gene that is toxic to bacteria, comprising the steps of:
(a) isolating genomic DNA from a culture of cells of a first bacterium, wherein said genomic DNA contains a gene that is toxic to a second bacterium;
(b) partially digesting said genomic DNA to produce genetic inserts, wherein at least one of said genetic inserts contains a gene that is toxic to said second bacterium;
(c) cloning said genetic inserts into a cloning vector;
(d) ligating together, under suitable ligation conditions, said genetic inserts and said cloning vector, to produce ligation products;
(e) transforming said ligation products directly into yeast; and
(f) amplifying said ligation products in said yeast.

2. The method of claim 1, wherein the first bacterium is Bacillus subtilis.

3. The method of claim 1, wherein the second bacterium is Escherichia coli.

4. The method of claim 1, wherein the genomic DNA is chromosomal DNA.

5. An expression library constructed according to the method of claim 1.

6. A method for screening for a gene that is toxic to bacteria, comprising the steps of:
(a) obtaining an expression library made in accordance with the method of claim 1; and
(b) screening said expression library for a gene that is toxic to bacteria.

7. The method of claim 6, wherein said expression library is screened using a mating strategy.
